(19) United States
(12) Patent Application Publication
(10) Pub. No.: US 2015/0045630 A1
(43) Pub. Date: Feb. 12, 2015

(54) PRESSURE MONITORING SYSTEM AND METHOD

(71) Applicants: Ruth Poliakine-Baruchi, Tel Aviv (IL); Lior Greenstein, Tel Aviv (IL); Amir Ben Shalom, Modiin (IL)

(72) Inventors: Ruth Poliakine-Baruchi, Tel Aviv (IL); Lior Greenstein, Tel Aviv (IL); Amir Ben Shalom, Modiin (IL)

(73) Assignee: ENHANCED SURFACE DYNAMICS, INC., Wellesley, MA (US)

(21) Appl. No.: 13/985,126

(22) PCT Filed: Feb. 23, 2012
(86) PCT No.: PCT/IB12/50829

§ 371 Date: Oct. 7, 2013

Related U.S. Application Data
(60) Provisional application No. 61/446,191, filed on Feb. 24, 2011.

(51) Int. Cl. A61B 5/00; A61B 5/11; A61B 5/0205

(52) U.S. Cl. CPC A61B 5/447; A61B 5/486; A61B 5/6892; A61B 5/6843; A61B 5/7405; A61B 5/743; A61B 5/7465; A61B 5/746; A61B 5/002; A61B 5/7475; A61B 5/0205; A61B 5/1121; A61B 5/024
USPC 600/301; 600/587; 600/483; 600/484; 600/595

(57) ABSTRACT

A pressure wound prevention system operable to prompt at least one caregiver to reposition a subject at risk of developing pressure wounds. A pressure detection apparatus comprising a plurality of sensors monitors pressure exerted upon the subject by a surface. A processor is operable to interpret and analyze data from the sensors and a notification mechanism such as a display, an alert, alarm or the like is provided to notify a caregiver when the subject requires repositioning.

### FIELD OF THE INVENTION

The embodiments disclosed herein relate to a pressure monitoring system and method, such as for preventing the development of pressure wounds, e.g. decubitus ulcers.

### BACKGROUND

Pressure wounds, e.g. decubitus ulcers, which are commonly known as pressure ulcers or bedsores, are lesions developed when a localized area of soft tissue is compressed between a bony prominence and an external surface for a prolonged period of time. Pressure ulcers may appear in various parts of the body, and their development is affected by a combination of factors such as unrelieved pressure, friction, shearing forces, humidity and temperature.

Currently, about 10%-15% of hospitalized patients are estimated to have bedsores at any one time (Source: Medicare website 2009). Although easily preventable and treatable if found early, bedsores are painful, and treatment is both difficult and expensive. In many cases bedsores can prove fatal—even under the auspices of medical care.

An effective way of dealing with pressure wounds is to prevent them. Existing preventive solutions are either passive (e.g., various types of cushioning) or active, including a range of dynamic mattresses that alternate the inflation/deflation of air cells. However, dynamic mattresses have a tendency to redistribute pressure from unnecessary locations thereby needlessly creating higher pressure in the sensitive areas, which may not effectively prevent pressure wounds.

A common preventive approach is keeping a strict routine of turning the patient every 2-3 hours or other determined time. This is a difficult, labor intensive and costly task and in many cases may be performed too late.

### SUMMARY

According to one embodiment a pressure wound prevention system is presented which is operable to prompt at least one caregiver to reposition a subject at risk of developing a pressure wound. The system may comprise: at least one pressure detection apparatus comprising a plurality of sensors configured to monitor pressure exerted upon the subject by a surface; at least one processor operable to interpret and analyze data from the sensors; and at least one notification mechanism configured to provide an alert to at least one caregiver when the subject requires repositioning.

In various embodiments the system may further comprise a timer and the notification mechanism may be triggered by the timer when an elapsed time has exceeded an elapsed time limit. Optionally, the elapsed time limit is determined according to data received from the sensors, automatically or manually as required.

Where appropriate, the system may comprise a timer override operable to pause and restart the timer. Accordingly, the timer override may be triggered to pause the timer when the pressure detection apparatus senses that the subject has left the surface. Additionally or alternatively, the timer override may be triggered to restart the timer when the pressure detection apparatus senses that the subject has returned to the surface.

Optionally, the system may further comprise a notification mechanism that may be customizable to suit requirements. Variously, the system may further comprise a notification override operable to disable the notification mechanism.

Variously, the notification mechanism may comprise an alert selected from a group consisting of: audio alerts, visual alerts, text alerts, graduated alerts or the like as well as combinations thereof.

Optionally, the notification mechanism may be configured to communicate with a remote control center via a wireless communication channel such as WiFi, Bluetooth, ZigBee or the like.

It is further noted that the system may further comprise a reset button via which the caregiver may reset the notification mechanism after repositioning the subject. Accordingly, where the pressure detection apparatus is configured to detect repositioning, the notification mechanism may be further operable to provide an alert if the reset button is activated without the subject being repositioned.

Where required the notification mechanism is further operable to prompt the caregiver to confirm that the subject has been repositioned. Accordingly, where the pressure detection apparatus is configured to detect repositioning, the notification mechanism may be further operable to provide an alert if the caregiver confirms that the subject has been repositioned without the subject being repositioned.

In some embodiments the pressure detection apparatus may be further configured to detect pressure from a plurality of body zones.

Furthermore, the system may further comprise a shutdown guard operable to shut down power to the system only if a caregiver enters an identification code.

Alternatively or additionally, the system may further comprise a reserve power storage unit for providing power to the system if a primary power supply is cut off.

Where appropriate, the system may further comprise a display unit configured to display pressure distribution data in a plurality of scales.

According to various embodiments the system may include auxiliary sensors for monitoring further parameters. For example, the pressure detection apparatus may comprise a weight sensor operable to measure a weight of the subject while supported upon the surface. Alternatively, or additionally, the pressure detection apparatus may comprise at least one vital sign sensor operable to monitor at least one of a heart rate or respiration of the subject.

Furthermore in still other embodiments the pressure detection apparatus may comprise at least one orientation sensor operable to monitor the angle of at least one body section of the subject.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1a is a schematic of the main components of a pressure monitoring system according to an embodiment;

FIG. 1b is a schematic of an extended pressure monitoring system according to an embodiment including a plurality of sub-systems;

FIGS. 2a-d depict isometric projections of multiple embodiments of a pressure-detection sheet;

FIG. 3 is a side cross-sectional view of an embodiment of a single sensor according to an embodiment;

FIG. 4a is a top plan view of another embodiment of a pressure detection sheet according to an embodiment;

FIG. 4b is a sectional view of the pressure detection sheet of FIG. 4a;

FIGS. 5a-d are representations of how pressure data may be displayed on a screen of a display system according to an embodiment; and

FIG. 5e shows a possible display screen for a notification mechanism indicating the orientation and pressure distribution for three sections of the subjects body.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale, and some features may be exaggerated or minimized to show details of particular components. Therefore, specific structural and functional details disclosed herein are not to be interpreted as limiting, but merely as a representative basis for teaching one skilled in the art to variously employ the present invention.

Embodiments described herein disclose a pressure monitoring system, such as for preventing the development of pressure wounds, the system configured to detect and monitor pressure created by a subject including at least one pressure detection sheet with a plurality of sensors configured to detect pressure, at least one driving unit configured to supply electrical potential to the sensors, at least one control unit configured to control the driving units and receive data from the sensors, at least one processor configured to interpret and analyze the data, and at least one display configured to present the data. The system may further include at least one storage unit configured to store data from control units and processors.

The pressure detection sheet may include an insulating layer sandwiched between a first conductive layer and a second conductive layer. According to one embodiment, each conductive layer has parallel strips of conductive material. Optionally, the first conductive layer and the second conductive layer are configured such that the parallel strips of the first conductive layer are arranged orthogonally to the parallel strips of the second conductive layer. Optionally, the parallel strips of the first conductive layer and the parallel strips of the second conductive layers overlap at a plurality of intersections. Optionally, these intersections form capacitance sensors.

In the pressure monitoring system according one embodiment, at least one display may be selected from a group comprising: computer screens, laptops, PDAs, cellular phone screens, printed sheets, and integrated LCD screens (e.g. TFT, touch screen). Optionally, the processor uses configurable parameters to analyze said data.

The system may further include at least one sensor configured to detect moisture. Optionally, the system further includes at least one sensor configured to detect temperature. Optionally, the pressure wound prevention system may be further configured to detect temperature and humidity. Optionally, the system comprises a plurality of pressure detection sheets in communication with a common control center.

In one or more embodiments, the pressure monitoring system may be further used to monitor the care routine of the subject. Optionally, the system may be further used as a data harvesting research tool.

Other embodiments include a method for pressure monitoring, such as for preventing the development of pressure wounds, wherein the method includes the steps of providing at least one pressure detection sheet comprising a plurality of sensors configured to detect pressure, supplying electrical potential to these sensors, receiving data from the sensors, interpreting and analyzing the data, and providing an output based upon the data.

Embodiments of the system and method for pressure monitoring provide a caregiver with indications of pressure distribution and ongoing, accumulated pressure exerted upon body parts of a patient, which may result in the creation or progression of a bedsore. A caregiver may then take proper action, such as to move the patient in a way that relieves pressure upon the effected body part. Embodiments of the system may also be used for ongoing analysis and recording of a patient's care routine.

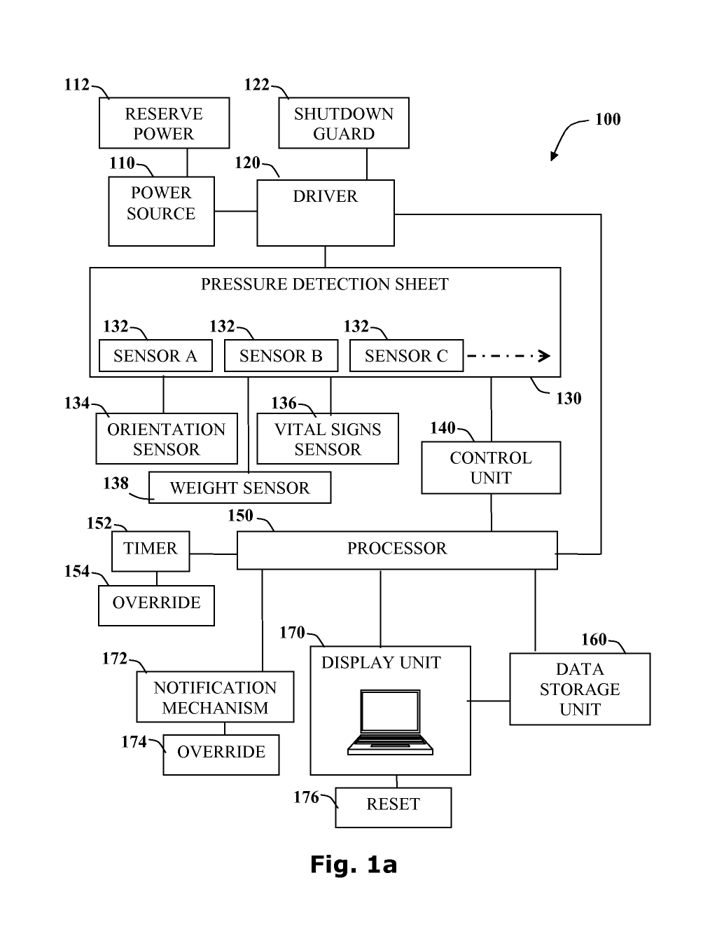

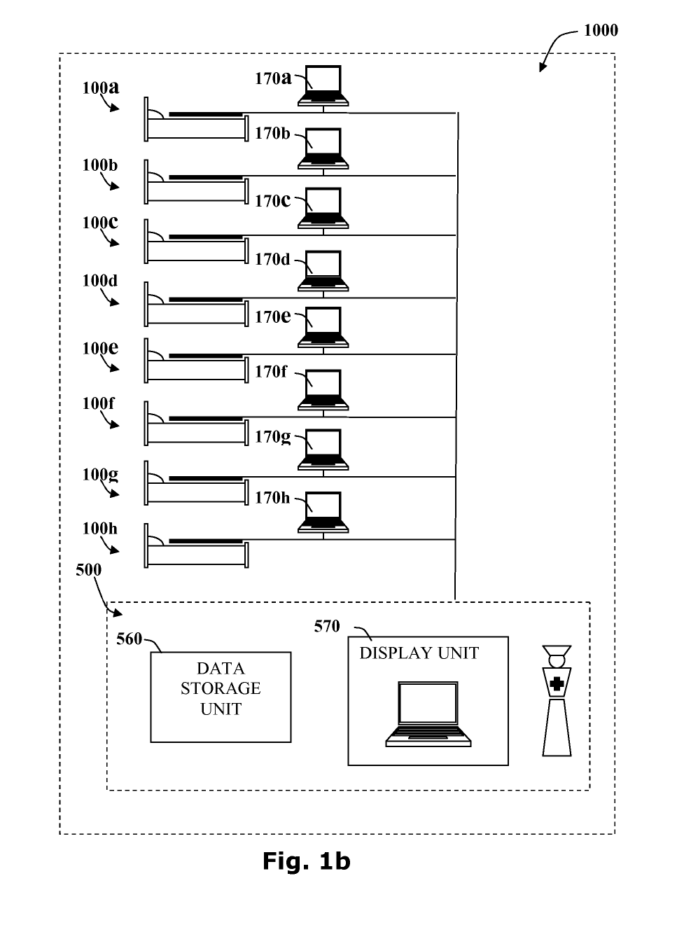

Reference is now made to the block diagram of FIG. 1a, showing an embodiment of a pressure monitoring system 100. The system may include at least one pressure-detection sheet 130 including a plurality of sensors 132, a driver 120, a control unit 140 which may be connected to a power source 110, a processor 150, a data storage unit 160 and a display system 170. Power may be supplied via a power cord connected to a wall outlet, or via battery power, optionally rechargeable. Battery support also allows for movement of the bed without requiring a powering off of the system. As a safety measure and for compliance tracking, caregiver authentication may be required via a shutdown guard 122 to confirm powering off the control unit 140, such as with entry of a caregiver's employee identification number.

In one embodiment, the driver 120 selectively supplies voltage to sensors in the pressure-detection sheet, the processor 150 monitors the potential across the sensors, calculates impedance values for each sensor, and stores that data in a data storage unit 160. The stored data may be further processed, analyzed, and displayed on a display system 170, such as computer screens, laptops, PDAs, cellular phone screens, printed sheets, integrated LCD screens (e.g. TFT, touch screen) and the like. Although presented in the block diagram of FIG. 1a as separate blocks, the system may optionally be integrated into a stand-alone system.

Referring now to FIG. 1b, an extended pressure monitoring system 1000 may include a plurality of sub-systems 100a-h in communication with a common remote control center 500. The sub-systems 100a-h may be, for example, beds in a hospital, care home or the like and may be configured to communicate with a remote control center 500, for example at a nursing station. This communication can be via wiring to a nurse call system, or alternatively via wireless communication (i.e., Bluetooth, ZigBee) to the nursing station. Alternatively, the plurality of sub-systems 100a-h may be located remotely from one another, for example each in an individual home, and the remote control center 500 may be a manned monitoring station.

The remote control center 500 may include a data storage unit 560 for storing data from the sub-systems 100a-h and a display unit 570 for presenting the data as required. The control center 500 may additionally provide processing and driving functionality for controlling multiple sub-systems. Optionally, each sub-system 100a-h may have its own dedicated monitor 170 for processing, storing and displaying data locally.

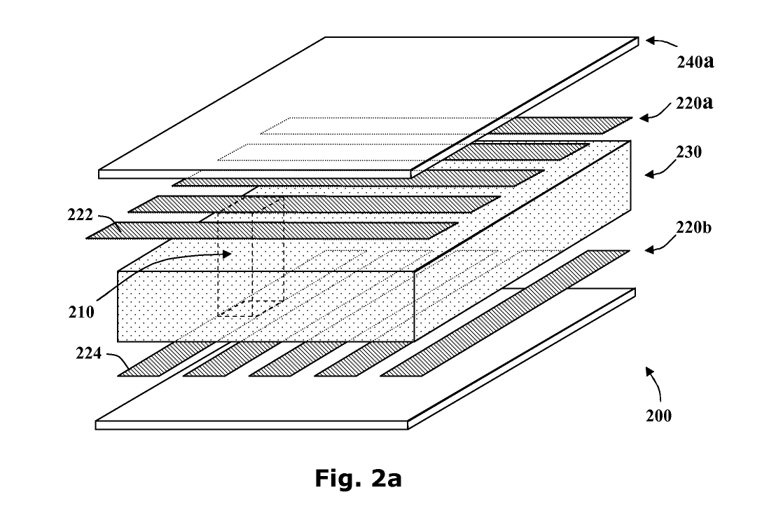

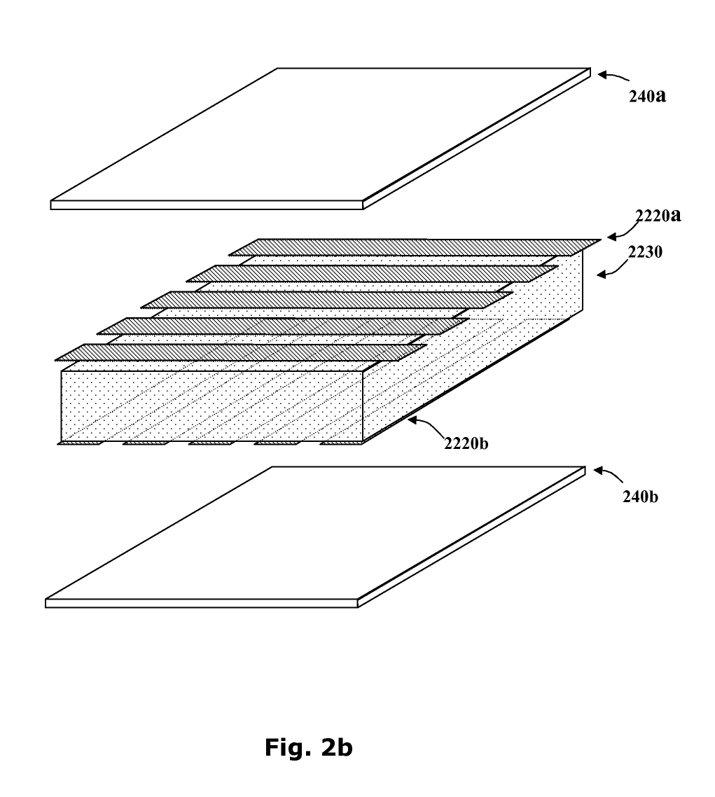

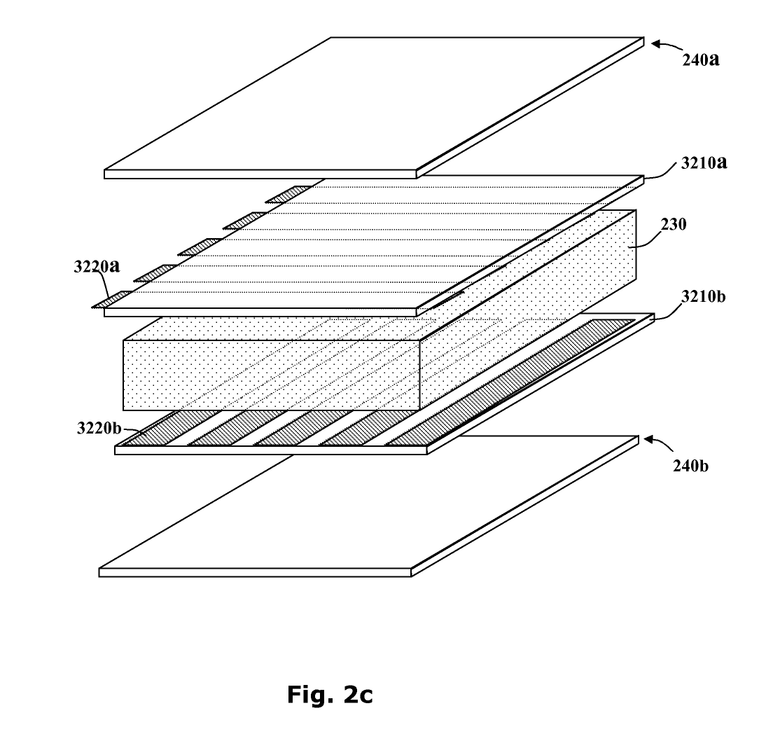

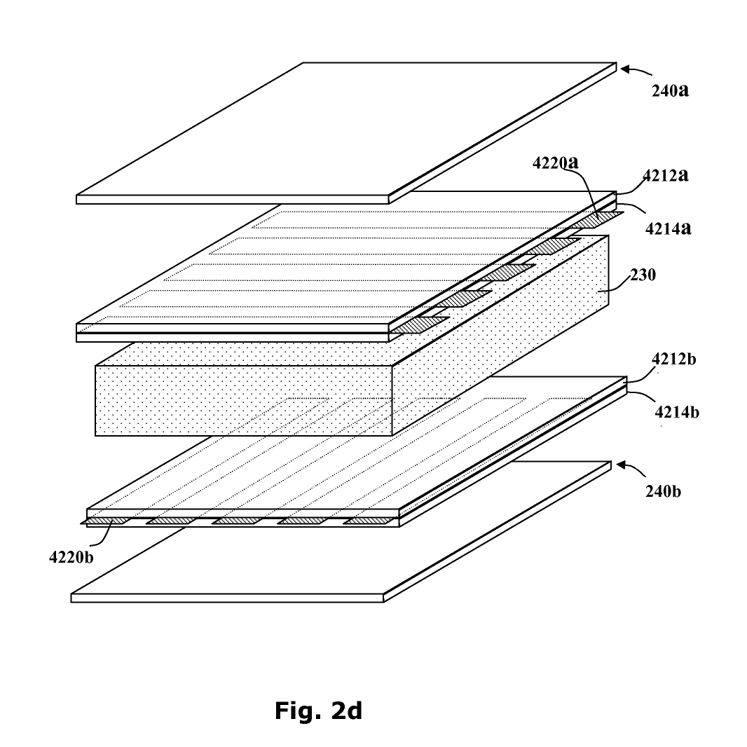

Reference is now made to FIG. 2a showing an embodiment of a pressure-detection sheet 200 comprising a plurality of sensors 210 arranged in a form of a matrix. The sheet may have two layers 220a, 220b of conductive material separated by an insulating layer 230 of isolating material. Each of the conductive layers may include parallel conductive strips 222, 224 and the two conductive layers 220a, 220b of strips 222, 224 may be arranged orthogonally such that in one conductive layer 220a the strips 222 are horizontal and in the other conductive layer 220b the strips 224 are vertical. Horizontal and vertical are used herein to describe the relative relationship of strips 222, 224 to one another, and these terms are not intended to be otherwise limiting. Each strip 222, 224 may be wired to a control unit and may operate under a low voltage source.

A capacitance sensor, such as that incorporated in pressure-detection sheet 200, may be based on the capacitance between the sections of the conducting strips 222, 224 overlapping at each “intersection” of a vertical conductive strip with a horizontal conductive strip. These capacitance sensors are configured such that pressing anywhere on their surface changes the spacing between the two conductive layers, and consequently the capacitance of the intersection. A driving unit may selectively provide an electric potential to the vertical strip and the electrical potential may be monitored on the horizontal strip such that the capacitance sensor of the overlapping section may be determined.

It is noted that by providing an oscillating electric potential across each sensor and monitoring the alternating current produced thereby, the impedance of the intersection may be calculated and the capacitance of the intersection determined. Thus, where the mechanical properties of the sensor are known, the pressure applied upon the sensor may be deduced.

The sheet 200 may further include additional sensors (not shown) configured to monitor additional factors, particularly those influencing the development of bedsores, such as temperature, humidity, or the like. Such additional sensors may be configured to monitor the factors continuously or intermittently as appropriate to detect high risk combinations of factors. Such measurements may be recorded and stored in a database for further analysis.

In some embodiments, the materials are selected such that the conductive layers 220a, 220b and insulating layers 230 are flexible. The insulation material may be a compressible sponge-like, airy or porous material (e.g., foam), allowing for a change in density when pressure is applied to it.

Referring back to FIG. 2a, the pressure-detection sheet 200, or sensing-mat, may be placed underneath or otherwise integrated with other material layers 240a, 240b such as used in standard bed sheets. The additional materials may confer further properties as needed for a particular application. The conductive material of the sensors may be wrapped by an isolating, water resistant, breathable cover sheet or the like, allowing minimum discomfort to the subject resting on the sheet.

With reference now to FIGS. 2b-d showing sections of other embodiments of the pressure-detection sheet, the conductive layers 220 (FIG. 2a) may be supported by various substrates. For example, FIG. 2b shows two conductive layers 2220a, 2220b adhered or otherwise attached to the insulating layer 230. Alternatively, as shown in FIG. 2c, conductive layers 3220a, 3220b may be supported by separate substrates 3210a, 3210b, for example thermoplastic polyurethane, the insulating layer 230 being sandwiched therebetween. In still another embodiment, as shown in FIG. 2d, the conductive layers 4220a, 4220b may themselves each be sandwiched between two substrates 4212a, 4214a, 4212b, 4214b, respectively.

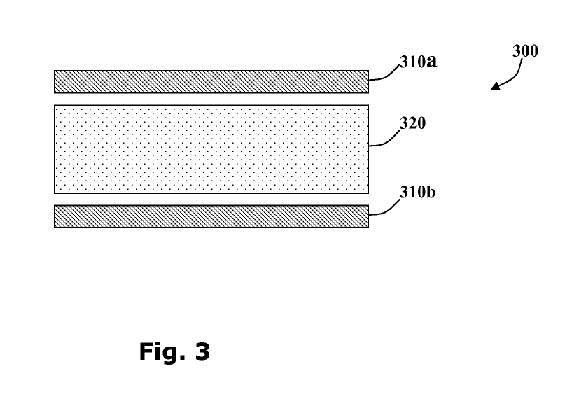

Reference is now made to FIG. 3, showing a cross section of an embodiment of a single sensor 300. The sensor may be a capacitor with two layers of conductive strips 310a, 310b and an insulating layer 320 of isolating material therebetween. Applying pressure to the sensor 300 would compress the insulating layer 320, changing the distance between the conductive strips 310a, 310b and thereby changing the capacitance of the capacitor.

In order to get an improved, stable reading of impedance values from a row of sensors, little or no movement should be made by the subject during the taking of readings from the sensors. Accordingly, in certain embodiments, the time taken for readings may be of the order of tens or hundreds of milliseconds, during which movement of the subject is generally insignificant. In applications where the subject is largely immobile, longer reading times may be used as required.

Referring back to FIG. 1, measurement readings from the multiple sensors 132 of the pressure-detection sheet 130 may be transmitted to a processor 150. Data transmission may be wireless or via data cables according to requirements. The processor 150 may be configured to interpret impedance values and to analyze the data to determine which sensors had pressure applied to them. The interpretation may be performed by consulting a lookup table, which maps impedance values at a given frequency to pressure values, often cited as units of millimeters of mercury (mm Hg), as commonly used in medical settings. Of course, it is understood that use of other units of pressure are also contemplated. The values in such a lookup table may differ from one sheet to another, and may need to be calibrated automatically or manually, either in the factory, upon initial usage of the sheet, or at specified points during the lifetime of the sheet. Impedance measurements may be affected by a number of properties of the sensors such as resistance, capacitance and inductance, any of which may indicate pressure according to the configuration of the sensing sheet.

The pressure-detection sheet or sensing mat 130 may be placed on surfaces such as a hospital bed, long term care facility bed, a home bed, a wheelchair, or the like. Embodiments of this system can detect the pressure points formed between a subject resting on the pressure-detection sheet 130 and the surface upon which the sheet rests. Pressure mapping data per subject may be aggregated over time in a data storage unit 160.

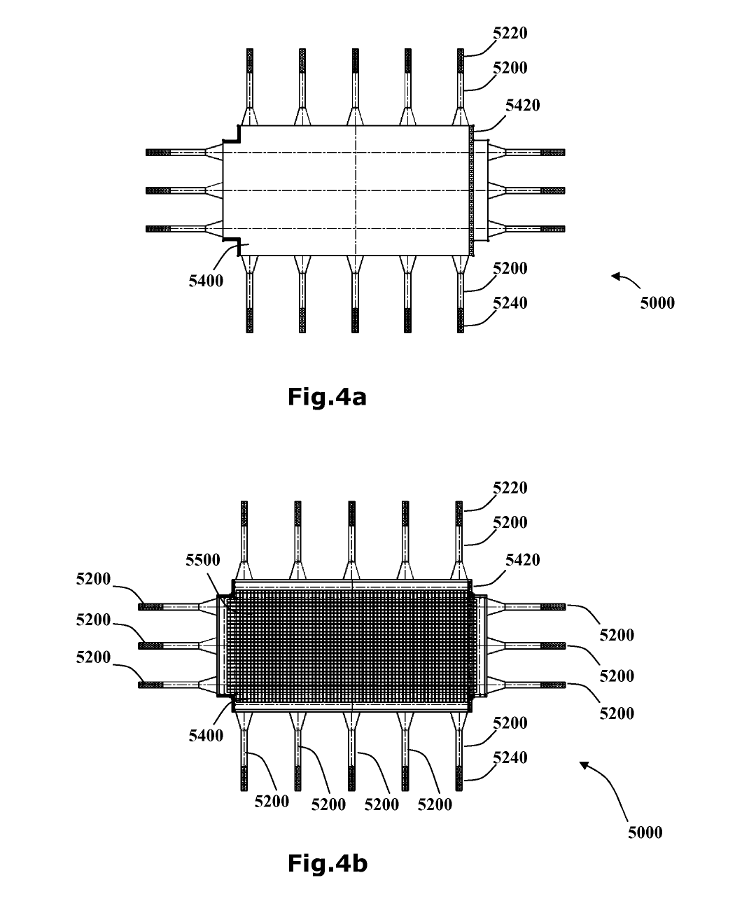

With reference to FIGS. 4a and 4b, a top view and cross-sectional view, respectively, are shown of a further embodiment of a pressure detection sheet 5000. The pressure detection sheet 5000 includes a sensor matrix 5500, such as described above, housed within a cover sheet 5400 and which may be sealed by a zipper 5420, or other fastener, as required.

The cover sheet 5500 may include attachment straps 5200 for tying the sheet to a mattress, bed or the like. Each attachment strap 5200 may have a coupling mechanism 5220 for attaching the straps to the mattress or to each other such that the pressure detection sheet 5000 is retained in position. This may be useful to prevent folding, wrinkling or other movement of the detection sheet 5000 which may contribute to the creation of shear forces which are known to encourage the formation of external pressure sores. Such a coupling mechanism 5220 may be for example hook and pile (e.g., Velcro®), buckles, adhesives, buttons, laces, clips, snaps, or others as is known in the art.

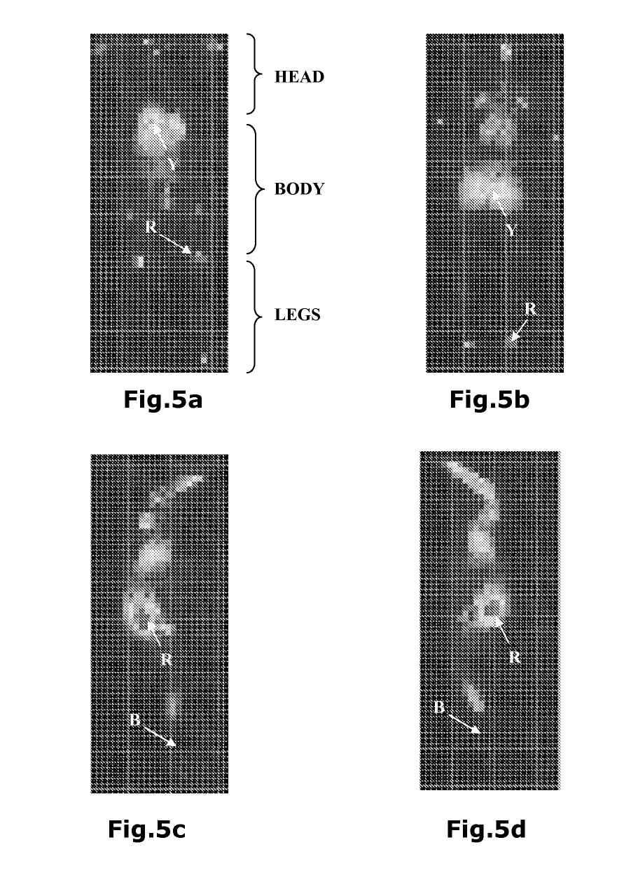

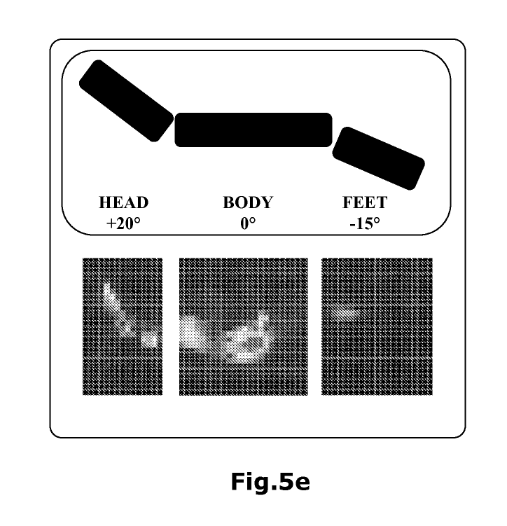

Reference is now made to the embodiments of FIGS. 5a-d, showing various representations of how pressure data may be displayed on a screen of a display system 170 (FIG. 1). Respectively, FIGS. 5a-d show the pressure distribution for a subject lying on his abdomen (FIG. 5a), his back (FIG. 5b), his left side (FIG. 5c) and his right side (FIG. 5d).

The display system 170 may be a computer in communication with the data storage unit 160, for example. Each display screen shows a matrix of pixels, each pixel representing one sensor of the pressure-detection sheet. The pressure detected by each pixel is represented by a visual indication. A grayscale may be used such that higher pressures are indicated by different shades, darker grays, for example. Alternatively or additionally, colors may be used, for example indicating high pressure formed between a subject's body and the surface on which the subject rests by displaying the pixel in a distinctive color, such as red (marked with R). Likewise, pixels representing sensors which detect low pressure or no pressure at all may be presented in other colors such as yellow (marked with Y), blue (marked with B) or black. It is understood that other colors or combinations are contemplated for the display screen 170. Furthermore, the ability to customize the pressure scale displayed is contemplated, such as for allowing pressure readings to be scaled up or down depending on the surface the patient is lying on. Such a feature may be useful, for example, to ensure that a caregiver is still alerted to body areas experiencing relatively high pressure even when the patient is lying on an airbed that lowers absolute pressure.

The display screen may identify different body zones, such as head, body, and foot sections of the pressure-detection mat, for example such as shown in the illustrative display of FIG. 5e. The identification of zones may allow pressure distribution to be more easily determined when a patient is in an unusual body position.

This arrangement may be useful, for example, where a patient is resting upon an adjustable bed consisting of a plurality of sections such as known in the art. By way of illustration, a hospital bed may be constructed from three or more sections which may each be configurable such that the patient adopts various postures. The pressure distribution over the sections maybe recorded and displayed, say, on a display showing the pressure distribution map for each section of the bed.

Furthermore, it is noted that by incorporating orientation sensors 134 (FIG. 1a) into the pressure-detection mat or the bed sections, the angle of each section of the bed and therefore of the patient's posture may be determined and accordingly displayed to a caregiver. Various orientation sensors may be used for the purpose of monitoring angle, for example a plurality of accelerometers may be incorporated into a pressure detection mat, an overlay, a coverlet, a mattress, a bed or the like to monitor angle of the section to the horizontal. Where appropriate, a three dimensional accelerometer may be used to provide information relating to lateral as well as longitudinal tilt of the surface.

Accordingly, the display may provide indication of the orientations of each monitored section of the subject's body. For example, a user interface may indicate that the head portion is orientated at an angle of +20 degrees to the horizontal, the body portion is orientated at an angle of 0 degrees to the horizontal, and the lower limbs portion is orientated at an angle of −15 degrees to the horizontal, say. Such indication may be presented as a text string as a map, by way or an icon such as shown in the illustrative display of FIG. 5e, or as may otherwise occur to those of the art.

It is particularly noted that information regarding the orientation and tilt of the body may be useful in the calculation of risk of a subject developing pressure wounds as a result of shear forces.

Furthermore, the display may additionally provide a notification that a care giver should reposition the subject in three dimensions, that is not only changing the two dimensional position of the subject upon the surface of the support but the orientation of the support itself. Indeed the display may be used to provide guidance to a care giver regarding how to reposition a patient based upon the patient's particular needs.

In various embodiments, the pressure-detection sheet 130 may include additional sensors which can be used to detect environmental parameters such as temperature, humidity, ambient pressure and the like. Still further, additional sensors 138 may be used to record a patient's weight, such that the patient does not have to be removed from the bed for weighing, or vital signs, such as heart rate and respiration. These additional sensors may be configured to send an alert to the caregiver.

Other embodiments of the pressure monitoring system can be designed for scale and stress, aiming to monitor the accumulated pressure on a plurality of subjects. Such embodiments may include a plurality of pressure-detection sheets connected to one or more drivers and control units. Power may be supplied from a plurality of sources, and single or multiple processors may be used for calculation and analysis of the data, which may be stored in one or a plurality of data storage units.

A software application may be used to retrieve data from the data storage unit, analyze it for different purposes, and may display the analysis results in various formats to a user. The software application may include features such as, but not limited to:
Calculating and presenting pressure detected by each sensor on a pressure-detection sheet;
Calculating shear forces pressures by comparing relative pressures detected by adjacent pixels or sensors;
Calculating and presenting the accumulated pressure over an elapsed time detected by each sensor on a pressure-detection sheet;
Calculating and presenting data such as temperature or moisture build-up over an elapsed time;
Calculating and alerting a caregiver at a monitoring station when patients need to be moved in order to prevent the creation of pressure wounds;
Calculating, presenting and alarming about different sheet parameters such as, but not limited to, wireless transmission malfunction, electricity disconnection, or the like;
Configuring parameters such as, but not limited to, pressure and time thresholds, for different patients or for different areas on the pressure-detection sheet;
Monitoring and logging a patient's pressure-relief care routine over time;
Monitoring caregivers' performances with regard to proper treatment of patients in their care;
Allowing visual and vocal alarms through a plurality of local and mobile devices and technologies such as, but not limited to, mobile phones, pagers, personal digital assistants (PDAs), display screens in nursing stations or medical carts, web interfaces, emails, Short Messaging Service (SMS), Multimedia Messaging Service (MMS), instant text messaging platforms and the like;
Allowing a caregiver to enter data with regard to patients' care status (for instance, when the patient was last moved); and
Allowing for presentation, monitoring, configuration, calculation, alarms and presentation of data from multiple sheets used by multiple subjects.

The system may provide several functions with respect to repositioning of a patient by a caregiver. In general, the system includes a user interface that includes a notification mechanism 172. As shown in FIG. 1a, the system may include a repositioning timer 152 in communication with the processor 150, and the user interface allows a caregiver to select an amount of time to elapse before a patient should be repositioned on the pressure-detection sheet in an effort to prevent the development of pressure wounds. The user interface may also provide an alert to a caregiver should pressure cease to be detected from the pressure-detection sheet, which may indicate that a patient has exited his/her bed. The system may further include a timer override 154, such that when a patient is removed from a bed under the authorization of a caregiver, the user interface may allow a caregiver to pause the repositioning timer and suspend bed exit alerts. When the system senses a patient placed back on the pressure-detection sheet, the system may restart the repositioning timer 152 and resume bed exit alerts, either automatically or upon receiving input from the caregiver via the user interface.

The system may identify a detected pressure increase or decrease prior to a designated time for repositioning a patient and provide an indication to a caregiver via the user interface. A caregiver may use such information to adjust the current repositioning time or a future repositioning time interval. More generally, the system may allow for a caregiver to customize the repositioning time based on individual patient need.

The system may prompt the caregiver to confirm that repositioning of a patient has been completed. A caregiver alert, such as a nurse call, can be automatically triggered upon or before the repositioning time elapsing, or can alternatively be disabled via a notification mechanism override 172. For example, disabling an audible alert may be desirable at night, allowing for “quiet time” when repositioning is not done as often. The system may sense unusual or improper repositioning of a patient and alert a caregiver of a potential exit attempt of the patient from a bed or warn of a potential impending fall from the bed. Furthermore, the system may receive confirmation of repositioning from a caregiver, such as by depressing a “reset button” 176 a “reposition” button or the like on the user interface, but determine via pressure detection that actual patient repositioning has not occurred and provide a corresponding alert.

Accordingly the system 100 may further comprise a reset button 176 via which the caregiver may reset the notification mechanism 172 after repositioning the subject. Where required the notification mechanism is further operable to prompt the caregiver to confirm that the subject has been repositioned. Where the pressure detection apparatus 130 is configured to detect repositioning, the notification mechanism 172 may be further operable to provide an alert if the caregiver presses the reset button 176 or otherwise confirms that the subject has been repositioned without the subject having been repositioned in practice.

It is further contemplated for the system and method described herein to enable storage of data collected from multiple subjects in a variety of situations and a plurality of locations. Data storage may be aggregated in one or more database units. Data storage may serve for statistics collection regarding a particular sheet or line of sheets, comparison of care settings according to patient groups (for instance diabetic patients), or for the creation of a research tool designed to provide practical recommendations for turning schedules and standard of care.

It will be appreciated that the system as described hereinabove may be particularly useful in care facilities such as, amongst others, acute care facilities, sub-acute care facilities, long term care facilities, home care environments, hospices, hospitals, nursing homes, assisted living facilities and the like. In addition, similar systems may be adapted for use in other environments such as bariatric beds, table/stretchers, hotel beds, vehicle seats, passenger seats, airplane seats, long-haul flight seats and the like.

The scope of the disclosed subject matter is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

1. A pressure wound prevention system operable to prompt at least one caregiver to reposition a subject at risk of developing a pressure wound, said system comprising:
at least one pressure detection apparatus comprising at least one layer of an insulating material sandwiched between a first layer of conducting strips and a second layer of conducting strips, said conducting strips of the first layer and said conducting strips of the second layer overlapping at a plurality of intersections, said plurality of intersections being a plurality of capacitive sensors configured to monitor pressure exerted upon said subject by a surface;
at least one processor operable to interpret and analyze data from said sensors; and
at least one notification mechanism in communication with the processor that is configured to provide an alert to said at least one caregiver when said subject requires repositioning.

2. The system of claim 1 further comprising a timer in communication with the processor wherein said notification mechanism is triggered by said timer when an elapsed time has exceeded an elapsed time limit.

3. The system of claim 2 wherein said elapsed time limit is determined according to data received from said sensors.

4. The system of claim 2 further comprising a timer override operable to pause and restart said timer.

5. The system of claim 4 wherein said timer override is triggered to pause said timer when said pressure detection apparatus senses that the subject has left the surface.

6. The system of claim 4 wherein said timer override is triggered to restart said timer when said pressure detection apparatus senses that the subject has returned to the surface.

7. (canceled)

8. The system of claim 1 further comprising a notification override operable to disable said notification mechanism.

9. The system of claim 1 wherein said notification mechanism comprises an alert selected from a group consisting of: audio alerts, visual alerts, text alerts, graduated alerts and combinations thereof.

10. The system of claim 1 wherein said notification mechanism is configured to communicate with a remote control center via a wireless communication channel.

11. The system of claim 1 further comprising a reset button via which the caregiver may reset the notification mechanism after repositioning the subject.

12. The system of claim 11 wherein said pressure detection apparatus is configured to detect repositioning and said notification mechanism is further operable to provide an alert if said reset button is activated without the subject being repositioned.

13. The system of claim 1 wherein said notification mechanism is further operable to prompt the caregiver to confirm that the subject has been repositioned.

14. The system of claim 13 wherein said pressure detection apparatus is configured to detect repositioning and said notification mechanism is further operable to provide an alert if the caregiver confirms that the subject has been repositioned without the subject being repositioned.

15. The system of claim 1 wherein said pressure detection apparatus is further configured to detect pressure from a plurality of body zones.

16. The system of claim 1 further comprising a shutdown guard operable to shut down power to the system only if a caregiver enters an identification code.

17. The system of claim 1 further comprising a reserve power storage unit for providing power to the system if a primary power supply is cut off.

18. The system of claim 1 further comprising a display unit configured to display pressure distribution data in a plurality of scales.

19. The system of claim 1 wherein the pressure detection apparatus further comprises a weight sensor operable to measure a weight of the subject while supported upon the surface.

20. The system of claim 1 wherein the pressure detection apparatus further comprises at least one vital sign sensor operable to monitor at least one of a heart rate or respiration of the subject.

21. The system of claim 1 wherein the pressure detection apparatus further comprises at least one orientation sensor operable to monitor the angle of at least one body section of the subject.
